(19) United States
(12) Patent Application Publication
(10) Pub. No.: US 2018/0194835 A1
(43) Pub. Date: Jul. 12, 2018

(54) USE OF VEGF ANTAGONIST IN TREATING CHORIORETINAL NEOVASCULAR AND PERMEABILITY DISORDERS IN PAEDIATRIC PATIENTS

(71) Applicant: Novartis AG, Basel (CH)

(72) Inventors: Gabriela Burian, Oberwil (CH); Sergey AKSENOV, Cambridge, MA (US)

(21) Appl. No.: 15/906,421

(22) Filed: Feb. 27, 2018

Related U.S. Application Data
(63) Continuation of application No. 14/903,749, filed on Jan. 8, 2016, now abandoned.
(60) Provisional application No. 61/845,064, filed on Jul. 11, 2013.

(51) Int. Cl. C07K 16/22; A61F 9/008; A61K 9/00; A61K 39/395; A61K 45/06

(52) U.S. Cl. CPC C07K 16/22; A61F 9/00821; A61K 9/0048; A61K 39/3955; A61K 45/06; A61K 2039/505; C07K 2317/76; C07K 2317/24; A61F 9/008

(57) ABSTRACT

The present invention relates to the use of a VEGF antagonist in the treatment of chorioretinal neovascular or permeability disorders in children. In particular, the invention provides a VEGF antagonist for use in a method for treating a child having CNV or ME, wherein said method comprises administering to the eye of a child a VEGF antagonist that either does not enter or is rapidly cleared from the systemic circulation. The VEGF antagonist may be administered intravitreally, e.g. through injection, or topically, e.g. in form of eye drops. The invention further provides the use of a VEGF antagonist in the manufacture of a medicament for treating a child having a chorioretinal neovascular or permeability disorder.

### TECHNICAL FIELD

This invention is in the field of treating retinal disorders in children.

### BACKGROUND ART

The growth of new blood vessels that originate from the choroid (the vascular layer of the eye between the retina and the sclera) and enter the sub-retinal pigment epithelium or subretinal space is referred to as “choroidal neovascularisation” (CNV). CNV in children can have a variety of aetiologies. For instance, CNV can be caused by inflammatory processes that may be triggered by an infectious agent. Examples are CNV secondary to (presumed) ocular histoplasmosis or toxoplasmosis, rubella retinopathy, sarcoidosis, toxocara canis, Vogt-Koyanagi-Harada syndrome and chronic uveitis. Other causes for CNV include traumatic choroidal rupture. CNV is also seen with retinal dystrophies which are often associated with an inherited genetic defect. Examples are CNV secondary to Best's disease, North Carolina macular dystrophy, Stargardt disease and choroideraemia. As in adults but more rarely, CNV in children has also been observed secondary to severe myopia, angioid streaks and choroidal osteoma. CNV in children can further be associated with optic nerve head drusen, optic nerve coloboma, and optic nerve pit and morning glory syndrome. In some cases, the underlying cause of CNV in children is unknown and therefore referred to as idiopathic CNV.

Standard treatments for CNV in adults include laser photocoagulation therapy (LPT), verteporfin (Visudyne®) photodynamic therapy (vPDT) and submacular surgery. Pharmacological treatment options are also available. For example, VEGF antagonists such as pegaptanib (Macugen®), ranibizumab (Lucentis®) and bevacimumab (Avastin®) have been used for treating CNV in adults.

Reports of the off-label use of pegaptanib, ranibizumab or bevacizumab in children are largely anecdotal (Kohly et al. (2011) Can J Ophthalmol 46(1):46-50). For example, two doses of pegaptanib led to almost complete retinal reattachment in a 2-year-old boy with stage 4 Coats' disease within three weeks after the treatment (Ciulla et al. (2009) Curr Opin Ophthalmol 20(3):166-74). Ranibizumab has been administered to children suffering from CNV secondary to keratoconus, interpapillomacular rupture of Bruch's membrane, ocular toxocariasis and Best's disease. Bevacizumab has been used to treat children suffering from CNV secondary to Coats' disease, myopia, choroidal osteoma, sensory retinal detachment due to blunt-force trauma to the eye, Best's disease, foveolar vitelliform lesion, choroidal rupture, toxoplasmosis, and cystoid macular oedema.

In some instances, combined treatments of bevacizumab with triamcinolone acetonide and/or LPT or vPDT have been used.

It is often difficult to predict how a drug successfully used in adults will behave in a paediatric population, especially in younger children (0-12 years). No adverse events have been observed in the cases reported to date when using antibody VEGF antagonists for treating CNV in children.

However, since ranibizumab and bevacizumab are usually administered intravitreally, some concerns have been voiced that a small amount of an antibody VEGF antagonist could enter the brain where it might interfere with a child's normal brain development (Sivaprasad et al. (2008) Br J Ophthalmol. 92:451-54). Potential concerns have also been raised with respect to the systemic exposure to an antibody VEGF antagonist when treating children (Lyall et al. (2010) Eye 24: 1730-31).

In addition, intravitreal administration is challenging in smaller children (below 6 years of age) as it usually requires general anaesthesia, which comes with an additional set of risk factors.

When CNV occurs secondary to a slowly progressing disease such as Best's disease, Coats' disease or severe myopia, beginning treatment early may be advantageous in preventing and delaying permanent damage to the retina and therefore may prevent or at least substantially delay vision loss. Similar considerations apply to CNV of other aetiologies because even a transient decrease in visual acuity can affect a child's normal development.

Vascular leakage leading to macular edema (ME) can result in irreversible structural damage and permanent loss of vision. ME is observed in conditions such as pseudophakic cystoid macular oedema (CME), uveitis-induced CME, trauma, sickle cell retinopathy etc. Congenital eye disorders such as Coats' disease can also increase the risk for developing ME early in life. The off-label use of intravitreal VEGF antagonists including bevacizumab as an adjunct in the management of Coats' disease in children has been reported (Kaul et al. (2010) Indian J Ophthalmol. 58(1):76-78, Cakir et al. (2008) J AAPOS 12(3):309-11).

No established standard of care for treating ME in children exists. Typical treatment options for ME include topical non-steroidal anti-inflammatory drugs (NSAID's), topical steroids, subscleral or intravitreal steroid treatment, laser photocoagulation and combinations of laser therapy and anti-inflammatory treatments.

It is thus an object of the invention to provide further and improved treatments for retinal disorders in children that address at least some of the current concerns regarding the treatment of children with antibody VEGF antagonists. In particular, the present invention relates to novel treatments and treatment schedules that are better suited for paediatric patients, e.g. by injecting a smaller dose and/or requiring fewer injections of a VEGF antagonist.

### DISCLOSURE OF THE INVENTION

The present invention relates to the use of a VEGF antagonist in the treatment of chorioretinal neovascular or permeability disorders in children. In particular, the invention provides a VEGF antagonist for use in a method for treating a child having CNV or ME, wherein said method comprises administering to the eye of a child a VEGF antagonist that either does not enter or is rapidly cleared from the systemic circulation. The VEGF antagonist may be administered intravitreally, e.g. through injection, or topically, e.g. in form of eye drops. The invention further provides the use of a VEGF antagonist in the manufacture of a medicament for treating a child having a chorioretinal neovascular or permeability disorder.

VEGF Antagonists

VEGF is a well-characterised signal protein which stimulates angiogenesis. Two antibody VEGF antagonists have been approved for human use, namely ranibizumab (Lucentis®) and bevacizumab (Avastin®). Ranibizumab and bevacizumab have shown great promise in treating ocular disease including CNV of various aetiologies in adults. The off-label use of ranibizumab or bevacizumab in children has been reported previously (see e.g. Kohly et al. (2011) Can J Ophthalmol 46(1):46-50).

While ranibizumab and bevacizumab have similar clearance rates from the eye into the blood stream, ranibizumab is excreted rapidly from the systemic circulation, whereas bevacizumab is retained and can suppress systemic VEGF levels for several weeks. More specifically, ranibizumab has a short systemic half-life of about 2 hours, whereas bevacizumab has a systemic half-life of about 20 days. In a developing organism like a child, this prolonged systemic VEGF suppression may have unwanted side effects on the normal development.

Therefore, in one aspect, the invention relates to the use of a VEGF antagonist in the treatment of a chorioretinal neovascular or permeability disorder in a child wherein the VEGF antagonist either does not enter or is rapidly cleared from the child's systemic circulation. In accordance with the invention, clearance of the VEGF antagonist may be sufficiently rapid when the systemic half-life of the VEGF antagonist is between 7 days and about 1 hour. Preferably, the systemic half-life of the VEGF antagonist of the invention is less than 7 days, more preferably less than 1 day, most preferably less than 3 hours. A preferred antibody VEGF antagonist is ranibizumab.

As an alternative, the VEGF antagonist is a non-antibody VEGF antagonist. Non-antibody antagonists include e.g. immunoadhesins. One such immunoadhesin with VEGF antagonist activity is aflibercept (Eylea®), which has recently been approved for human use and is also known as VEGF-trap (Holash et al. (2002) PNAS USA 99:11393-98; Riely & Miller (2007) Clin Cancer Res 13:4623-7s). Aflibercept has a systemic half-life of around 5-6 days and is the preferred non-antibody VEGF antagonist for use with the invention. Aflibercept is a recombinant human soluble VEGF receptor fusion protein consisting of portions of human VEGF receptors 1 and 2 extracellular domains fused to the Fc portion of human IgG1. It is a dimeric glycoprotein with a protein molecular weight of 97 kilodaltons (kDa) and contains glycosylation, constituting an additional 15% of the total molecular mass, resulting in a total molecular weight of 115 kDa. It is conveniently produced as a glycoprotein by expression in recombinant CHO K1 cells. Each monomer can have the following amino acid sequence (SEQ ID NO: 1):

SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSCMHEALHNHYTQKSLSLSPG

and disulfide bridges can be formed between residues 30-79, 124-185, 246-306 and 352-410 within each monomer, and between residues 211-211 and 214-214 between the monomers.

Another non-antibody VEGF antagonist immunoadhesin currently in pre-clinical development is a recombinant human soluble VEGF receptor fusion protein similar to VEGF-trap containing extracellular ligand-binding domains 3 and 4 from VEGFR2/KDR, respectively, and domain 2 from VEGFR1/Flt-1; these domains are fused to a human IgG Fc protein fragment (Li et al. (2011) Molecular Vision 17:797-803). This antagonist binds to isoforms VEGF-A, VEGF-B and VEGF-C. The molecule is prepared using two different production processes resulting in different glycosylation patterns on the final proteins. The two glycoforms are referred to as KH902 (conbercept) and KH906. The fusion protein can have the following amino acid sequence (SEQ ID NO:2):

MSVYWDTGVLLCALLSCLLLTGSSSGGRPFVEMYSEIPEIIHMTEGRELV
IPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTAR
TELNVGIDFNWEYPSSKHQHKKLVNRDLKTQSGSEMKKFLSTLTIDGVTR
SDQGLYTCAASSGLMTKKNSTFVRVHEKPFVAFGSGMESLVEATVGERVR
LPAKYLGYPPPEIKWYKNGIPLESNHTIKAGHVLTIMEVSERDTGNYTVI
LTNPISKEKQSHVVSLVVYVPPGPGDKTHTCPLCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKATP
PVLDSDGSFFLYSKLTVDKSRWQQGNVFSCSVMHEALHNHYTQKSLSLSP
GK

and, like VEGF-trap, can be present as a dimer. This fusion protein and related molecules are further characterized in EP1767546.

Other non-antibody VEGF antagonists include antibody mimetics. (e.g. Affibody® molecules, affilins, affitins, anticalins, avimers, Kunitz domain peptides, and monobodies) with VEGF antagonist activity. Due to their small size, antibody mimetics are typically cleared from the circulation rapidly (within minutes to hours). Pegylation is one way used to extend local and systemic half-life.

Therefore the term “non-antibody VEGF antagonists” includes recombinant binding proteins comprising an ankyrin repeat domain that binds VEGF-A and prevents it from binding to VEGFR-2. One example for such a molecule is DARPin® MP0112. The ankyrin binding domain may have the following amino acid sequence (SEQ ID NO: 3):

GSDLGKKLLEAARAGQDDEVRILMANGADVNTADSTGWTPLHLAVPWGHL
EIVEVLLKYGADVNAKDFQGWTPLHLAAAIGHQEIVEVLLKNGADVNAQD
KFGKTAFDISIDNGNEDLAEILQKAA

Recombinant binding proteins comprising an ankyrin repeat domain that binds VEGF-A and prevents it from binding to VEGFR-2 are described in more detail in WO2010/060748 and WO2011/135067. Pegylation extends the systemic half-life of DARPins® to 1-3 days.

Further specific antibody mimetics with VEGF antagonist activity are the 40 kD pegylated Anticalin® PRS-050 (Mross et al. (2011) Molecular Cancer Therapeutics 10: Supplement 1, Abstract A212) and the monobody pegdinetanib (also referred to as Angiocept or CT-322, see Dineen et al. (2008) BMC Cancer 8:352).

The afore-mentioned non-antibody VEGF antagonist may be modified to further improve their pharmacokinetic properties. For example, a non-antibody VEGF antagonist may be chemically modified, mixed with a biodegradable polymer or encapsulated into microparticles to increase intravitreal retention of and reduce systemic exposure to the non-antibody VEGF antagonist.

Variants of the above-specified VEGF antagonists that have improved characteristics for the desired application may be produced by the addition or deletion of amino acids. Ordinarily, these amino acid sequence variants will have an amino acid sequence having at least 60% amino acid sequence identity with the amino acid sequences of SEQ ID NO: 1, SEQ ID NO: 2 or SEQ ID NO: 3, preferably at least 80%, more preferably at least 85%, more preferably at least 90%, and most preferably at least 95%, including for example, 80%, 81%, 82%, 83%, 84%, 85%, 86%, 87%, 88%, 89%, 90%, 91%, 92%, 93%, 94%, 95%, 96%, 97%, 98%, 99%, and 100%. Identity or homology with respect to this sequence is defined herein as the percentage of amino acid residues in the candidate sequence that are identical with SEQ ID NO: 1, SEQ ID NO: 2 or SEQ ID NO; 3, after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a standard scoring matrix such as PAM 250 can be used in conjunction with the computer program (see Dayhoff et al. (1978) Atlas of Protein Sequence and Structure, vol. 5, supp. 3). For example, the percent identity can then be calculated as: the total number of identical matches multiplied by 100 and then divided by the sum of the length of the longer sequence within the matched span and the number of gaps introduced into the shorter sequences in order to align the two sequences.

If a non-antibody VEGF antagonist is used in practising the invention, the non-antibody VEGF antagonist binds to VEGF via one or more protein domain(s) that are not derived from the antigen-binding domain of an antibody. The non-antibody VEGF antagonist is preferably proteinaceous, but may include modifications that are non-proteinaceous (e.g., pegylation, glycosylation). In some embodiments of the invention, the VEGF antagonist of the invention preferably does not comprise the Fc portion of an antibody as the presence of the Fc portion in some instances increases the half-life of the VEGF antagonist and extends the time the VEGF antagonist is present in circulation.

Pegylation

Due to their small size, antibody mimetics are typically cleared from the circulation rapidly (within minutes to hours). Thus, in some embodiments of the invention, in particular where the VEGF antagonist is an antibody mimetic, one or more polyethylene glycol moieties may be attached at different positions in the VEGF antagonist molecule.

Such attachment may be achieved by reaction with amines, thiols or other suitable reactive groups. The thiol group may be present in a cysteine residue; and the amine group may be, for example, a primary amine found at the N-terminus of the polypeptide or an amine group present in the side chain of an amino acid, such as lysine or arginine.

Attachment of polyethylene glycol (PEG) moieties (pegylation) may be site-directed. For instance, a suitable reactive group may be introduced into the VEGF antagonist to create a site where pegylation can occur preferentially. For example, a VEGF antagonist such an antibody mimetic (e.g. DARPin® MP0112) may be modified to include a cysteine residue at a desired position, permitting site directed pegylation on the cysteine, for example by reaction with a PEG derivative carrying a maleimide function. Alternatively, a suitable reactive group may already originally be present in the VEGF antagonist.

The PEG moiety may vary widely in molecular weight (i.e. from about 1 kDa to about 100 kDa) and may be branched or linear. Preferably, the PEG moiety has a molecular weight of about 1 to about 50 kDa, preferably about 10 to about 40 kDa, even more preferably about 15 to about 30 kDa, and most preferably about 20 kDa. For example, addition of a PEG moiety of 20 kDa has been shown to extend the half-life of DARPin® in circulation to up to 20 hours, while larger PEG moieties of 40 to 60 kDa in size increased circulatory half-life to about 50 hours.

Patient

The present invention relates to the use of a VEGF antagonist in treating chorioretinal neovascular or permeability disorders in children. A patient is considered to be a child when he or she has not yet completed his or her 18th year of life. In one embodiment, a child according to the invention is older than 1 year but less than 18 years old.

At the age of 12 years, the human eye is essentially fully developed. Intravitreal administration of a VEGF antagonist to children of 12 years of age or above is therefore not expected to interfere with the normal development of the eye. Because of the lack of data and the theoretical risks of administration of an inhibitor of VEGF, which is involved in many of the pathways necessary for growth and development (angiogenesis, endothelial differentiation and development of the blood-brain barrier), it is considered a higher risk to administer a VEGF antagonist to children less than 12 years.

In a particular embodiment, the child is less than 12 years of age. The child may be 5 years old or older, but less than 12 years of age. In yet another embodiment, a child is older than 1 year (e.g. 2 years or older) but less than 5 years old. Administering a VEGF antagonist to a younger child in need thereof may outweigh the risks of systemic exposure to the antagonist where permanent visual impairment or complete vision loss is virtually unavoidable in the absence of the treatment.

Indications

The present invention relates to the treatment of a chorioretinal neovascular or permeability disorder in a child. Chorioretinal neovascular or permeability disorders observed in children include CNV and ME.

CNV treatable by the present invention may be secondary to a variety of diseases and disease processes that occur in children. For example, diseases that cause inflammation in the eye may lead to CNV. Such diseases include ocular histoplasmosis or toxoplasmosis, rubella retinopathy, sarcoidosis, toxocara canis, Vogt-Koyanagi-Harada syndrome and chronic uveitis. In paediatric patients with a history of the aforementioned infectious diseases associated with the subsequent development of CNV, in particular (presumed) ocular histoplasmosis, toxoplasmosis and toxocara canis, treatment with a VEGF antagonist according to the invention should start at the first sign of CNV to prevent or delay permanent damage to the retina.

A further cause of CNV is retinal dystrophy. Early onset retinal dystrophies are associated with one or more gene defect(s). Examples are Best's disease, North Carolina macular dystrophy, Stargardt disease and choroideraemia Coats' disease may also have a hereditary component and is likewise associated with CNV. In accordance with the invention, treatment of CNV with a VEGF antagonist may be particularly favourable in children with Best's disease and/or Coats' disease. In paediatric patients having a family history of and therefore an increased risk for developing retinal dystrophy, treatment with a VEGF antagonist according to the invention should start at the first sign of CNV to prevent or delay permanent damage to the retina. For children with Best's disease, treatment may be initiated before the child reaches the age of 10, preferably before the age of 6. For children with Coats' disease, treatment may be initiated early on, preferably at stage I of Coats' disease which is characterised by telangiectasia only.

CNV may occur secondarily to damage to the choroid after a physical insult. For example, choroidal rupture may occur due to trauma to the eye.

Choroidal tumours may also be associated with CNV. Tumour growth can result in an acute decrease in vision due to serous macular detachment or a subretinal haemorrhage and may include CNV. A rare and benign choroidal tumour is choroidal osteoma.

CNV treatable by the present invention therefore includes CNV associated with or secondary to a variety of conditions including post-traumatic choriopathy, angioid streaks/pseudoxanthoma elasticum, Best's disease, central serous chorioretinopathy, punctate inner choriopathy, multifocal choroiditis, histoplasmosis syndrome, choroidal osteoma, toxoplasmosis, uveitis, pseudotumor cerebri, peripapillary, idiopathic choriditis, pathologic myopia, polypoidal choroidal vasculopathy, and central serous chorioretinopathy.

Retinal neovascularisation treatable by the present invention includes retinal neovascularization secondary to sickle cell retinopathy, retinal angiomatous proliferation, ROP, and Coats' disease.

ME treatable by the present invention may be associated with or secondary to pseudophakia, uveitis, occlusive vasculitis, retinitis pigmentosa, branch retinal vein occlusion (BRVO), central retinal vein occlusion (CRVO), ocular ischemic syndrome, radiation optic neuropathy/retinopathy, post-inflammatory choroidal neovascularisation, proliferative diabetic retinopathy (PDR), sickle cell retinopathy, Eales disease, or nonarteritic ischemic optic neuropathy.

Other chorioretinal neovascular or permeability disorders that may be treatable by the present invention further include choroidal metastatic diseases, melanoma associated neovascularization, macroaneurysm, vasoproliferative tumour, juxtapapillary capillary hemangioma, idiopathic macular teleangiectasis, herpetic corneal neovascularization, cicatricial pemphigoid corneal neovascularization, posterior capsular neovascularization, dry eye-associated corneal neovascularization, bleb revision, adjunct glaucoma filtering surgery, neovascular glaucoma and idiopathic CNV.

Dosing

Ranibizumab is typically administered to adults intravitreally at a dose of 0.5 mg in a 50 μl volume. Aflibercept is also administered via intravitreal injection. The typical adult dose is 2 mg (suspended in 0.05 ml buffer comprising 40 mg/ml in 10 mM sodium phosphate, 40 mM sodium chloride, 0.03% polysorbate 20, and 5% sucrose, pH 6.2).

Children who are at least 12 years old typically receive the same dose of the VEGF antagonist that is administered to an adult. While growth and development of the eye continue beyond the age of 12 years, the size of the eye in children of this age group is comparable to the average size of the eye in adults and therefore the serum exposure to an intravitreally administered VEGF antagonist is not expected to be much higher than that observed in adults. In addition, the body has reached a developmental stage in which it is more similar to the body of an average adult.

However, the normal dose and/or volume may be reduced for the treatment of younger children (below the age of 12, in particularly below the age of 5) due the reduced intravitreal volume of their eyes, smaller body weight and the increased risks for the body's normal development associated with systemic VEGF antagonist exposure.

In one embodiment, only the VEGF antagonist dose is reduced (e.g. to reduce systemic VEGF antagonist exposure), while the administered volume is kept the same. Dose reduction can be achieved by diluting an adult formulation through the addition of a sterile, buffered solution (ideally the same buffer in which the VEGF antagonist is provided in the adult formulation). Smaller volumes are sometimes harder to manage and may result in greater variation of the amount of VEGF antagonist actually administered to a patient. Therefore in some embodiments, the VEGF antagonist dose is reduced without reducing the volume that is used to administer the VEGF antagonist. For example, the dose may be reduced but the volume may be kept similar to the typical adult volume (e.g. by giving 0.24 mg ranibizumab dose in a 40 μl volume using a 6 mg/ml formulation).

In other embodiments, the same dose is administered, but in a reduced volume (to account for the smaller size of the eye in children below 12 years of age).

Preferably, both the dose and the volume are reduced. Typically, both the dose and the volume administered to children below the age of 12 and above the age of 1 year are 60% or less of the typical dose and volume of a VEGF antagonist administered to an adult. The dose and the volume may be reduced proportionally to the reduced intravitreal volume of the eye according to the child's age in order to maintain the same ocular concentration that have been found to be efficacious in adults.

In some instances, however, reducing the dose proportionally to the reduced intravitreal volume of a child's eye may not be sufficient to prevent systemic VEGF antagonist exposure levels that exceed those that were found to be safe in the adult population. Systemic exposure is correlated to the body weight of the subject. Therefore, when choosing specific doses for the administration to children, the possibility of underexposure relative to the reference adult vitreal exposure (decreased efficacy) needs to be balanced against the increased serum exposure (increased risk). Hence, in some embodiments of the invention, the dose administered to a child is reduced further than what would be dictated by a proportional reduction relative to the reduced intravitreal volume of the child's eye in order to maintain safe systemic VEGF antagonist exposure levels.

Existing formulations of a VEGF antagonist may be used to achieve the reduced doses and volumes. A 10 mg/ml formulation of ranibizumab is particularly suitable to provide doses and volumes adapted for different age and patient groups (e.g. 0.5 mg, 0.4 mg, 0.3 mg, 0.25 mg, 0.2 mg, 0.15 mg 0.1 mg or 0.05 mg in 50 μl, 40 μl, 30 μl, 25 μl, 20 μl, 15 μl, 10 μl and 5 μl, respectively). Similarly, a 6 mg/ml formulation of ranibizumab can be used to administer 0.06 mg, 0.12 mg, 0.18 mg and 0.24 mg in 10 μl, 20 μl, 30 μl and 40 μl, respectively.

In accordance with the invention, children in the 5 to 12-year age group may receive about 60% of the typical adult dose in about 60% of the typical adult volume (e.g. 0.3 mg ranibizumab in a 30 μl volume). Alternatively, the dose may be halved but the volume may be reduced only slightly (e.g. by administering 0.24 mg ranibizumab in a 40 μl volume).

Children below the age of 5 years, but older than 1 year, may receive about 40% of the typical adult dose in about 40% of the typical adult volume. For example, 0.2 mg ranibizumab may be administered in a 20 μl volume.

In some instances, the dose can be increased to achieve efficacy. For example, the dose in children older than 1 year and younger than 5 years may be increased to half or slightly more than half of the adult dose (e.g. for ranibizumab 0.25 mg in 25 μl or 0.3 mg in 30 μl). However, for children in this age group, the dose typically should not exceed 60% of the typical adult dose to avoid exposure to serum levels of the VEGF antagonist well above levels that have been found to be safe in adults. Preferably, the dose administered to children in this age group should not exceed 50% of the typical adult dose.

Similarly, the dose in children older than 5 years and younger than 12 years may be increased to about three quarters of the adult dose (e.g. for ranibizumab 0.4 mg in 40 μl). However, for children in this age group, the dose typically should not exceed 80% of the typical adult dose to avoid exposure to serum levels of the VEGF antagonist well above those levels that have been found to be safe in adults. Preferably, the dose administered to children in this age group should not exceed 70% of the typical adult dose.

Administration

The VEGF antagonist of the invention will generally be administered to the patient via intravitreal injection, though other routes of administration may be used, such as a slow-release depot, an ocular plug/reservoir or eye drops. Administration in aqueous form is usual, with a typical volume of 5-50 μl e.g. 7.5 μl, 10 μl, 15 μl, 20 μl, 25 μl, or 40 μl. Injection can be via a 30-gauge×½-inch (12.7 mm) needle.

In some instances, an intravitreal device may be used to continuously deliver a VEGF antagonist into the eye over a period of several months before needing to be refilled by injection. When a VEGF antagonist is administered continuously, the dose and the release-rate can be adjusted using the ocular and systemic exposure models described herein. Preferably, the intravitreal device is designed to release the VEGF antagonist at an initial rate that is higher in the first month. The release rate slowly decreases, e.g., over the course of the first month after implantation, to a rate that is about 50% less than the initial rate. The container may have a size that is sufficient to hold a supply of the VEGF antagonist that lasts for about four to six months. Since a reduced dose of VEGF antagonist may be sufficient for effective treatment when administration is continuous, the supply in the container may last for one year or longer, preferably about two years, more preferably about three years.

Continuous delivery of a VEGF antagonist may be more suitable in children who are 12 years of age or older since the eye has essentially reached its adults size. Where implantation of an intravitreal device interferes with the normal development of a child's eye, continuous delivery may not be suitable. For example, continuous delivery of a VEGF antagonist may not be suitable in children less than 12 years old, particularly in children less than 5 years old, more particularly in children less than 2 years old.

Various intravitreal delivery systems are known in the an. These delivery systems may be active or passive. For example, WO2010/088548 describes a delivery system having a rigid body using passive diffusion to deliver a therapeutic agent. WO2002/100318 discloses a delivery system having a flexible body that allows active administration via a pressure differential. Alternatively, active delivery can be achieved by implantable miniature pumps. An example for an intravitreal delivery system using a miniature pump to deliver a therapeutic agent is the Ophthalmic MicroPump System™ marketed by Replenish, Inc. which can be programmed to deliver a set amount of a therapeutic agent for a pre-determined number of times.

For continuous administration, the VEGF antagonist is typically encased in a small capsule-like container (e.g. a silicone elastomer cup). The container is usually implanted in the eye above the iris. The container comprises a release opening. Release of the VEGF antagonist may be controlled by a membrane positioned between the VEGF antagonist and the opening, or by means of a miniature pump connected to the container. Alternatively, the VEGF antagonist may be deposited in a slow-release matrix that prevents rapid diffusion of the antagonist out of the container.

Continuous administration via an intravitreal device may be particularly suitable for patients with chronic CNV secondary to, e.g., angioid streaks, central serous chorioretinopathy, Vogt-Koyanagi-Harada syndrome, or pseudoxanthoma elasticum. Patients with CNV refractory to conventional treatment with anti-inflammatory therapy may also be benefit from continuous administration. Because only a small surgery is required to implant a delivery system and intravitreal injections are avoided, patient compliance issues with repeated intravitreal injections can be avoided. Intravitreal concentrations of the VEGF antagonist are reduced, and therefore the potential risk of side-effects from VEGF antagonist entering the circulation is decreased. Avoiding intravitreal injections may be particularly advantageous in children who may require general anaesthesia for intravitreal injections. Systemically elevated VEGF antagonist levels may interfere with normal growth and development of children who therefore may benefit from lower intravitreal concentrations of the VEGF antagonist.

In one aspect of the invention, the VEGF antagonist is provided in a pre-filled sterile syringe ready for administration. Preferably, the syringe has low silicone content. More preferably, the syringe is silicone free. The syringe may be made of glass. Using a pre-filled syringe for delivery has the advantage that any contamination of the sterile VEGF antagonist solution prior to administration can be avoided. Pre-filled syringes also provide easier handling for the administering ophthalmologist.

In accordance with the invention, a pre-filled syringe will contain a suitable dose and volume of a VEGF antagonist of the invention. Typically, both the dose and the volume in the pre-filled syringe is 60% or less of the typical dose and volume of a VEGF antagonist administered to an adult. A typical volume of VEGF antagonist in the pre-filled syringe is 5-50 μl, e.g. 7.5 μl, 10 μl, 15 μl, 20 μl, 25 μl, or 40 μl. For example, a pre-filled syringe may contain a 10 mg/ml formulation of ranibizumab (e.g. comprising 0.4 mg, 0.3 mg, 0.2 mg or 0.1 mg in 40 μl, 30 μl, 20 μl and 10 μl, respectively).

Alternatively, a prefilled syringe may contain a 6 mg/ml formulation of ranibizumab (e.g. comprising 0.06 mg, 0.12 mg, 0.18 mg and 0.24 mg in 10 μl, 20 μl, 30 μl and 40 μl, respectively).

In a preferred embodiment, a pre-filled low-dose syringe in accordance with the invention has a nominal maximal fill volume of 0.2 ml and is specifically adapted to accurately dispense volumes below 5011.

In another aspect of the invention, the VEGF antagonist is provided as part of a kit. In addition to a container comprising the VEGF antagonist, the kit will further comprise a syringe. The syringe is used for intravitreal administration of the VEGF antagonist. Preferably, the syringe is a low-dose syringe, i.e. a syringe that measures small volumes with high accuracy. In some embodiments, the container will comprise more than one dose of the VEGF antagonist and more than one syringe allowing the use of the kit for multiple administrations of the VEGF antagonist.

Slow-Release Formulations

VEGF antagonist may be provided as slow-release formulations. Slow-release formulations are typically obtained by mixing a therapeutic agent with a biodegradable polymer or encapsulating it into microparticles. By varying the manufacture conditions of polymer-based delivery compositions, the release kinetic properties of the resulting compositions can be modulated. Addition of a polymeric carrier also reduces the likelihood that any intravitreal administered VEGF antagonist enters the circulation or reaches the developing brain of a child.

A slow-release formulation in accordance with the invention typically comprises a VEGF antagonist, a polymeric carrier, and a release modifier for modifying a release rate of the VEGF antagonist from the polymeric carrier. The polymeric carrier usually comprises one or more biodegradable polymers or co-polymers or combinations thereof. For example, the polymeric carrier may be selected from poly-lactic acid (PLA), poly-glycolic acid (PGA), poly-lactide-co-glycolide (PLGA), polyesters, poly (orthoester), poly(phosphazine), poly (phosphate ester), polycaprolactones, or a combination thereof. A preferred polymeric carrier is PLGA. The release modifier is typically a long chain fatty alcohol, preferably comprising from 10 to 40 carbon atoms. Commonly used release modifiers include capryl alcohol, pelargonic alcohol, capric alcohol, lauryl alcohol, myristyl alcohol, cetyl alcohol, palmitoleyl alcohol, stearyl alcohol, isostearyl alcohol, elaidyl alcohol, oleyl alcohol, linoleyl alcohol, polyunsaturated elaidolinoleyl alcohol, polyunsaturated linolenyl alcohol, elaidolinolenyl alcohol, polyunsaturated ricinoleyl alcohol, arachidyl alcohol, behenyl alcohol, erucyl alcohol, lignoceryl alcohol, ceryl alcohol, montanyl alcohol, cluytyl alcohol, myricyl alcohol, melissyl alcohol, and geddyl alcohol.

In a particular embodiment, the VEGF antagonist is incorporated into a microsphere-based sustained release composition. The microspheres are preferably prepared from PLGA. The amount of VEGF antagonist incorporated in the microspheres and the release rate of the VEGF antagonist can be controlled by varying the conditions used for preparing the microspheres. Processes for producing such slow-release formulations are described in US 2005/0281861 and US 2008/0107694.

The need and extent for dose and release-rate adjustment for a slow-release formulation suitable for administration to children can be assessed using the ocular and systemic exposure models described herein.

Treatment Regimens

In accordance with the invention, the VEGF antagonist is administered one or more times initially and then re-administered “as needed” depending on the effectiveness of the initial course of treatment. In some embodiments, the initial treatment is limited to a single intravitreal injection of the VEGF antagonist.

By performing additional injections on an “as needed” basis, spacing between administrations of the VEGF antagonist after the initial treatment may be increased as a second or further dose of the VEGF antagonist is administered only when signs of disease activity can be observed by the treating physician. Exposure to high serum levels of VEGF antagonist is therefore further reduced. In addition, reducing the total number of required injections decreases the risk of other potential adverse events, e.g. due to general anaesthesia that may be needed for safe administration of the antagonist to younger children. Performing intravitreal injections less frequently may also increase patient compliance resulting in an overall more effective treatment. This is particularly advantageous in patients suffering from CNV secondary to a slowly progressing retinal degenerative disease such as Stargardt disease or Best's disease who may require multiple injections over an extended period of time to improve visual acuity or prevent vision loss. Reducing the total number of administrations also results in a more cost-effective therapy.

In some instances, a single injection of the VEGF antagonist according to the invention may be sufficient to ameliorate the disease or prevent disease progression for many years. In other instances, one, two or three injections, each at least one month apart are administered to the patient, while any subsequent injections are performed less frequently, preferably on an “as needed” basis. In some embodiments, the injections are at least 6 weeks, preferably 8 weeks, more preferably 10 weeks apart.

Treatment may be discontinued when maximum visual acuity is achieved. For example, treatment may be discontinued when visual acuity is stable for at least three months (i.e., no increase or decrease in visual acuity is observed during this period).

Administration in an individualised “as needed” regimen is based on the treating physician's judgment of disease activity. Disease activity may be assessed by observing the change in best corrected visual acuity (BCVA) from baseline (i.e. from the initial dose of VEGF antagonist) over time, starting at month 1, and up to month 12 after the first administration of VEGF antagonist. In addition or alternatively, changes in disease activity are assessed by observing changes in clinical and anatomical signs in response to the treatment.

For example, the VEGF antagonist is administered to a patient the first time after an initial diagnosis of a chorioretinal neovascular or permeability disorder (e.g. CNV or ME) has been made (typically as a consequence of the patient becoming visually impaired or during routine examination in patients predisposed to developing such a disorder). The diagnosis can be made during examination of the eye by a combination of slit-lamp evaluation, biomicroscopic fundus examination, ophthalmoscopy, optical coherence tomography (OCT), fluorescein fundus angiography (FFA) and/or colour fundus photography (CFP).

The spacing of follow-up examinations is typically at the discretion of the treating physician. For example, follow-up examinations may take place every four weeks or more after the initial administration of the VEGF antagonist (e.g. monthly or bimonthly). For example, follow-up examinations may take place every 4-6 weeks, every 6-8 weeks, every 8-10 weeks etc.

A second, third or further administration of the VEGF antagonist is performed only if examination of the eye reveals signs of a persistent or recurring chorioretinal neovascular or permeability disorder during a follow-up examination. The interval between injections should not be shorter than one month. During the follow-up examinations, CNV and ME lesion activity parameters (such as active angiogenesis, exudation and vascular leakage characteristics) can be assessed on the basis of imaging results of OCT, FFA, CFP etc. and/or clinical assessment (including BCVA). Changes in these parameters are recorded over time, typically starting at month 1, and up to month 12, after the initial dose of VEGF antagonist has been administered.

Changes in key anatomical parameters of the CNV and ME lesions (e.g. reduced retinal thickness or fluid leakage) indicate a reduction of disease activity. BCVA improvements of ≥5, ≥10, or ≥15 letters at month 6 and month 12 compared to baseline are also indicative of treatment success. In these cases, no further administrations of the VEGF antagonist may be needed. A loss in BCVA of ≥5, ≥10, or ≥15 letters from baseline or sustained disease activity (e.g. no reduction in retinal thickness, continued leakage as indicated by the presence of fluid) indicates the need for one or more additional injections of the VEGF antagonist.

Combination Therapy

The compounds of the invention may be administered in combination with one or more additional treatment(s).

In one aspect of the invention, treatment with a VEGF antagonist of the invention may be used in combination with LPT or vPDT.

LPT uses laser light to cause controlled damage of the retina to produce a beneficial therapeutic effect. Small bursts of laser light can seal leaky blood vessels, destroy abnormal blood vessels, seal retinal tears, or destroy abnormal tissue in the back of the eye. It is quick and usually requires no anaesthesia other than an anaesthetic eye drop. LPT techniques and apparatuses are readily available to ophthalmologists. See Lock et al. (2010) Med J Malaysia 65:88-94

LPT techniques can be classified according to their application as focal, panretinal (or scatter), or grid. Focal LPT applies small-sized burns to specific points of focal leakage (i.e. microaneurysms). Panretinal LPT scatters burns throughout the peripheral retina. Grid LPT applies a pattern of burns to areas of the retina with diffuse capillary leakage or non-perfusion, with each burn typically spaced apart by two visible burn widths. Patients can receive more than one type of LPT (e.g. a combination of focal and panretinal LPT) and these may be administered one directly after the other, or after a delay. A typical therapeutic panretinal LPT involves the application of 1200-1600 burns.

Laser spot sizes (spot diameters) of 50-500 μm are typical (smaller spot sizes are more usual for focal LPT, larger for panretinal), applied for 50-200 ms (continuously, or via micropulses), using green-to-yellow wavelengths e.g. using an argon gas (514.5 nm) laser, a krypton yellow laser (568.2 nm), or a tunable dye laser (variable wavelength). In some cases a red laser may be used if a green or yellow laser is precluded (e.g. if vitreous hemorrhage is present).

Micropulse laser therapy (MLP) uses 810 nm or 577 nm lasers to direct a discontinuous beam of laser light on the affected tissue (Kiire et al. (2011) Retina Today, 67-70). This results in a greater degree of control over the photothermal effects in laser photocoagulation. The steady continuous-wave emission of conventional LPT is delivered in form of short laser pulses. Each pulse typically is 100-300 μs in length with a 1700 to 1900 μs interval between each pulse. The “width” (“ON” time) of each pulse and the interval between pulses (“OFF” time) are adjustable by the surgeon. A shorter micropulse “width” limits the time for the laser-induced heat to spread to adjacent tissue. A longer interval between pulses allows cooling to take place before the next pulse is delivered. Intraretinal damage thus can be minimised. Hence MLP is also referred to as “sub-threshold laser treatment” or “tissue-sparing laser therapy”. 10-25% of micropulse power is sufficient to show a consistent photothermal effect that is confined to the retinal pigment epithelium and does not affect the neurosensory retina.

According to the invention, patients can receive both LPT and a VEGF antagonist. Administration of LPT and the VEGF antagonist should not occur simultaneously, so one will precede the other. The initiation of LPT and of VEGF antagonist administration occur within 6 months of each other, and ideally occur within 1 month of each other (e.g. within 10 days).

Typically, VEGF antagonist therapy is administered prior to LPT. LPT can take place promptly after VEGF antagonist administration (e.g. within 2-20 days, typically within 10-14 days), or can take place after a longer delay (e.g. after at least 4 weeks, after at least 8 weeks, after at least 12 weeks, or after at least 24 weeks). Injected VEGF antagonists are expected to maintain significant intravitreal VEGF-binding activity for 10-12 weeks (Stewart & Rosenfeld (2008) Br J Ophthalmol 92:667-8). In an alternative embodiment, the VEGF antagonist therapy is administered after LPT.

Some embodiments involve more than one administration of LPT and/or of VEGF antagonist. For instance, in one useful embodiment a patient receives in series (i) VEGF antagonist, (ii) at least one administration of LPT, (iii) VEGF antagonist. For instance, the patient may receive an initial intravitreal injection of a VEGF antagonist; then, within 10-14 days of receiving the VEGF antagonist, he or she receives focal LPT, followed by a second injection of the VEGF antagonist at least 4 weeks or a month after the initial injection. Alternatively, within 10-14 days of receiving a VEGF antagonist, a patient may receive at least one sitting (e.g. up to three) of panretinal LPT; and then, 4 weeks or a month after the initial injection, the patient receives a second injection of the VEGF antagonist. This regimen may be continued with further doses of the VEGF antagonist, e.g. with a frequency of every 1 or 2 months or as needed. By ensuring that LPT is initiated within 14 days of the initial injection, the antagonist will still be present in the eye.

Combining VEGF antagonist therapy with LPT is particularly useful for treating extrafoveal and juxtafoveal CNV in teenagers and older cooperative children (e.g. 6 years and older) because similar techniques as those used in adults can be applied. Juxtafoveal treatment of CNV by LPT is not recommended in smaller children (less than 6 years of age) due to the high risk of an inadvertent foveal burn.

vPDT uses a light-activated molecule to cause localised damage to neovascular endothelium, resulting in angioocclusion. Light is delivered to the retina as a single circular spot via a fiber optic cable and a slit lamp, using a suitable ophthalmic magnification lens (“cold” laser light application). The light-activated compound—verteporfin (Visudyne®)—is injected into the circulation prior to the laser light application, and damage is inflicted by photoactivation of the compound in the area afflicted by CNV. Verteporfin is transported in the plasma primarily by lipoproteins. Once verteporfin is activated by light in the presence of oxygen, highly reactive, short-lived singlet oxygen and reactive oxygen radicals are generated which damages the endothelium surrounding blood vessels. Damaged endothelium is known to release procoagulant and vasoactive factors through the lipo-oxygenase (leukotriene) and cyclooxygenase (eicosanoids such as thromboxane) pathways, resulting in platelet aggregation, fibrin clot formation and vasoconstriction. Verteporfin appears to somewhat preferentially accumulate in neovasculature. The wavelength of the laser used for photoactivation of the light-activated compound may vary depending on the specific light-activated compound used. For example, 689 nm wavelength laser light delivery to the patient 15 minutes after the start of the 10-minute infusion with verteporfin may be used. Photoactivation is controlled by the total light dose delivered. When using vPDT in the treatment of CNV, the recommended light dose is 50 J/cm² of neovascular lesion administered at an intensity of 600 mW/cm² over 83 seconds. Light dose, light intensity, ophthalmic lens magnification factor and zoom lens setting are important parameters for the appropriate delivery of light to the predetermined treatment spot during vPDT and may need to be adapted depending on the laser system used for therapy.

Administration of the VEGF antagonist is performed before or after vPDT. Typically, administration of the VEGF antagonist and vPDT will be performed on the same day. Typically, intravitreal injection of the VEGF antagonist is performed last to minimise the handling of the eye after injection. Alternatively, treatment with VEGF antagonist is initiated at least 1 week, 2 weeks, 3 weeks, 4 weeks, 2 months, 3 months, 4 months, 5 months or 6 months before vPDT. The VEGF antagonist may be administered every 4 weeks, every 6 weeks, or every 8 weeks. Treatment may be continued at the same interval or extended intervals after vPDT. Where the interval is extended, the period between administration of the VEGF antagonist may increase by 50% or 100%. For example, if the initial interval was 4 weeks, the interval may be extended to 6 or 8 weeks. Alternatively, VEGF antagonist administration may be continuous, for example, if an intravitreal delivery system is used. The intravitreal device may be implanted prior to vPDT. Alternatively, a single administration of non-antibody VEGF antagonist shortly before or after vPDT may be sufficient to achieve the desired effect. For example, a single dose of VEGF antagonist may be given on the day of the vPDT.

vPDT is preferably administered only once but may be repeated as needed. Generally, vPDT is not given more frequently than every 3 months, vPDT may be repeated every 3 months. Alternatively, vPDT may be repeated less frequently, in particular if the VEGF antagonist treatment is continued after vPDT. Typically, vPDT is administered on an “as needed” basis. Ideally, continued treatment with a VEGF antagonist treatment after vPDT prevents recurrence of CNV.

vPDT has been used as monotherapy or in combination with an anti-inflammatory agent in children and usually requires only one session to improve visual acuity. However, pronounced alterations of the retinal pigment epithelium were reported in a number of cases. In one embodiment, vPDT is less preferred as part of a combination therapy with a VEGF antagonist for the treatment of CNV in children. Combination of vPDT with triamcinolone can result in increased intraocular pressure. Therefore combining VEGF therapy with vPDT and triamcinolone should be avoided.

In a further aspect of the invention, treatment time and patient compliance is improved by using a VEGF antagonist in combination with an anti-inflammatory agent. Administering the VEGF antagonist in combination with an anti-inflammatory agent can have synergistic effects depending on the underlying cause of CNV. Addition of an anti-inflammatory agent is particularly advantageous in CNV secondary to an inflammatory disease or condition. Anti-inflammatory agents include steroids and NSAIDs. NSAIDs used in the treatment of ocular diseases include ketorolac, nepafenac and diclofenac. In some instances, the use of diclofenac is preferred. Corticosteroids used in treating ocular diseases include dexamethasone, prednisolone, fluorometholone and fluocinolone. Other steroids or derivatives thereof that may be used in combination with VEGF antagonist treatment include anecortave, which has angiostatic effects but acts by a different mechanism than the VEGF antagonists according to the invention. A preferred anti-inflammatory agent is triamcinolone. The anti-inflammatory agent may also be a TNF-α antagonist. For example, a TNF-α antibody may be administered in combination with a non-antibody VEGF antagonist. TNF-α antibodies, e.g. those sold under the trade names Humira®, Remicade®, Simponi® and Cimzia®, are well known in the art. Alternatively, a TNF-α non-antibody antagonist such as Enbrel® may be administered in combination with a VEGF antagonist.

The anti-inflammatory agent may be administered at the same time as the VEGF antagonist. The anti-inflammatory agent can be administered either systemically or locally. For example, the anti-inflammatory agent may be administered orally, topically, or, preferably, intravitreally. In a specific embodiment, triamcinolone is administered intravitreally at the same time as the VEGF antagonist of the invention.

In yet another aspect of the invention, the VEGF antagonist is administered after administration of an antimicrobial agent. For example, the antimicrobial agent may be selected from gatifloxacin, ciprofloxacin, ofloxacin, norfloxacin, polymixin B+chloramphenicol, chloramphenicol, gentamicin, fluconazole, sulfacetamide, tobramycin, neomycin+polymixin B. and netilmicin. Alternatively, the antimicrobial agent may be selected from pyrimethamine, sulfadiazine and folinic acid or a combination thereof. Combination with pyrimethamine can be particularly advantageous in treating patient with CNV associated with toxoplasmosis.

General

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

### DESCRIPTION OF THE DRAWINGS

FIG. 1: Predicted exposure ratios of the maximum serum concentration (Cmax) of ranibizumab in children receiving single bilateral intravitreal ranibizumab doses of 0.1-0.5 mg relative to the reference in vitro IC₅₀=11 ng/ml. Predicted ranges of exposure represent uncertainty in model assumptions.

FIG. 2: Predicted exposure ratios for the area under the curve (AUC) of ranibizumab in the serum (black) and vitreous (grey) of children receiving single bilateral intravitreal ranibizumab doses of 0.1-0.5 mg relative to the reference AUC of ranibizumab in the serum of adults receiving a single unilateral intravitreal ranibizumab dose of 0.5 mg. Predicted ranges of exposure represent uncertainty in model assumptions.

### MODES FOR CARRYING OUT THE INVENTION

### Comparative Example 1

A 13-year-old boy presented with a 4-week history of floaters in his right eye associated with a 5-day history of blurred vision. Visual acuity was counting fingers. Eye examination showed a mild anterior chamber reaction and vitritis. Fundal examination showed a pale area of chorioretinitis inferotemporal to the disc with surrounding serous elevation involving the fovea and peripapillary region. Left eye examination was normal. Further investigation revealed that the patient had played with his pet dog 4 weeks previously before eating sweets without washing his hands. A diagnosis of toxocara chorioretinitis was made. The patient was treated with prednisolone 60 mg. Vision improved initially to 0.0 (Log MAR). However, after tapering the dose, vision deteriorated to 0.5. Fundal examination showed juxtafoveal subretinal haemorrhage. CNV was diagnosed by fluorescein angiography.

An intravitreal injection of ranibizumab was administered to the patient. At 1 month, his vision had improved to 0.0. A continued small area of leakage was observed during a follow-up visit. After two further injections each 1 month apart, no further leakage from the membrane and resolution of subretinal fluid was observed by fluorescein angiography and ocular coherence tomography. The patient's visual acuity remained stable at −0.2 at the 12-month follow-up.

### Comparative Example 2

This non-randomized, retrospective case series was designed to investigate the long-term safety and efficacy of off-label intravitreal bevacizumab (IVB) for the treatment of pediatric retinal and choroidal diseases other than retinopathy of prematurity (ROP). Patients younger than 18 years of age treated with IVB between Jan. 1, 2005 and Jan. 1, 2013 were included in the study. Exclusion criteria included a follow up of less than 6 months, a history of ROP, and eyes presenting with light perception or worse vision.

From one hundred and four eyes treated with IVB for pediatric retinal and choroidal diseases, 81 eyes of 77 patients were included in the current study. Average age was 9.1 years (range: 8 months to 17 years) and 45/77 (58%) patients were male. Patients received a mean number of 4.1 injections (range 1-17) and average follow up was 788 days. Primary diagnoses of patients treated with IVB included Coats' disease (n=30), choroidal neovascular membrane (n=27), familial exudative vitreoretinopathy (FEVR, n=13), cystoid macular edema (n=5), and other (n=6). Average Snellen equivalent visual acuity at presentation was 20/228 and improved to 20/123 at 6 months (p=0.017) and 20/108 at 12 months follow up (p=0.002). Average central foveal thickness improved from 439 microns at presentation to 351 microns at 6 months (p=0.005) and 340 microns at 12 months (p<0.001). Statistically significant visual acuity gains at 12 months were seen in patients with choroidal neovascular membrane (p=0.013), but visual acuity gains did not reach statistical significance for cystoid macular edema (p=0.06), Coats' disease (p=0.14) or FEVR (p=0.54). The only systemic adverse event identified in the current study was the development of idiopathic intracranial hypertension in an obese 16-year-old female with FEVR. Adverse ocular side effects included ocular hypertension (IOP>30) requiring topical therapy in 8 eyes of 7 patients, of which 5 eyes were on concomitant local or topical corticosteroid therapy. Worsening of tractional retinal detachment was seen in 2 eyes with FEVR.

Patients receiving IVB for the treatment of pediatric retinal and choroidal diseases other than ROP experienced significant visual acuity gains and reductions in central macular thickness. IVB was well-tolerated with minimal side effects noted at a mean follow up of 788 days.

### Example 1

A Pharmacokinetic Model for Predicting the Ocular and Systemic Exposure to Intravitreally Administered Ranibizumab in Children

To model the ocular and systemic exposure to ranibizumab in children, two key relationships were established based on published data:
1. A relationship between the age of a child and the vitreous chamber depth and density of the vitreal gel to predict the ocular clearance rate and vitreal concentration;
2. A relationship between age and body weight of a child, and PK parameters of systemic disposition (allometric scaling) to predict the systemic concentration.

Vitreal concentration of ranibizumab was calculated using the volume of the vitreous body. It was calculated as the volume of a partial sphere whose height equals the vitreous chamber depth (VCD) and whose diameter equals the axial length (AL) of the eye. The VCD of children and adults was age-correlated using a piecewise linear regression model and published data for children up to 3 years old (Fledelius & Christensen (1996) Br J Ophthalmol 80(10):918-921); older children (Twelker et al. (2009) Optom Vis Sci 86(8):918-935); and adults (Neelam et al. (2006) Vision Res 46(13):2149-2156). The AL of the eye in each age group was calculated using an aspect ratio equal to the ratio of the average AL and VCD values obtained from the publications cited above.

Ocular clearance rate of ranibizumab in the human eye was calculated using a one-dimensional model of diffusion and convection in a porous medium (Zhao & Nehorai (2006) IEEE Trans Signal Process 54(6):2213-2225; Dechadilok & Deen (2006) Ind Eng Chem Res 45(21):6953-6959). In this model, the eye is represented as a cylinder whose axis of symmetry coincides with the posterior-anterior axis of the eye. The front side of the cylinder is the hyaloid membrane next to the anterior chamber, and the back side of the cylinder is the retina. The length of the cylinder equals the VCD. In addition to the VCD, the ocular clearance rate in this model is determined by the density of the vitreal gel. A relationship between vitreal density and ocular clearance rate was established using published data (Tan et al. (2011) Invest Ophthalmol Vis Sci, 52(2):1111-1118). The relationship between age and vitreal density was based on published information (Oyster (1999) The Human Eye, Sinauer Associates Incorporated, pp. 530-544). The model was further calibrated to match the ocular kinetics established in adults for intravitreally administered ranibizumab (the Novartis population PK model of ranibizumab).

Systemic disposition of ranibizumab was described using a population PK model (the Novartis population PK model of ranibizumab). The relationship between body weight and systemic clearance was modelled using standard allometric scaling principles (Anderson. & Holford (2008) Annu Rev Pharmacol Toxicol 48(1):303-332). The body weight of children and adults was calculated using established relationships between age and parameters of the body weight distribution (Portier et al (2007) Risk Anal 27(1):11-26).

Model simulations were performed for typical patients and provided an expected average exposure. Typical children were modelled to be 2, 5, 12 or 18 years old. A typical adult was modelled to be 70 years old.

Exposure was simulated for a range of those key model parameters which are expected to impact the predicted exposure the most. Exponents of allometric scaling relationships between systemic clearance and volume of distribution and body weight were varied between 0.37-0.75 (clearance) and 0.41-1 (volume). Potentially greater permeability of the immature ocular membranes in young children was captured by increasing the ocular clearance rate by 50% relative to the adult value.

### Example 2

### Ranibizumab Dose Determination for Treating Children with Chorioretinal Neovascular and Permeability Disorders

Using the pharmacokinetic model described in Example 1, the predicted ocular and systemic exposure in children receiving intravitreally administered ranibizumab was compared to the exposure in adults following intravitreal injection of 0.5 mg ranibizumab, since the efficacy and safety profiles for adults at this dose level and mode of administration are known.

Exposure ratios to ranibizumab were calculated for three different parameters: (i) the maximum concentration (Cmax) in serum, which provides a measure of acute toxicity, (ii) the area under the curve (AUC) in serum, which provides a measure of potential long-term toxicity associated with continual inhibition of systemic VEGF, and (iii) the AUC in the vitreous which provides a measure of efficacy associated with continual inhibition of VEGF in the eye.

The ratio of predicted exposure in children to exposure in adults represents a measure of likelihood of ocular and systemic toxicity and can be used to determine the relative benefit/risk ratio of paediatric doses. Doses with a systemic exposure ratio of less than 1 are considered to have an acceptable safety profile. The serum concentration should also be lower than the in vitro IC₅₀ for ranibizumab which is in the range of 11-27 ng/ml. Doses with a vitreous exposure ratio close to 1 are considered to have an acceptable efficacy profile.

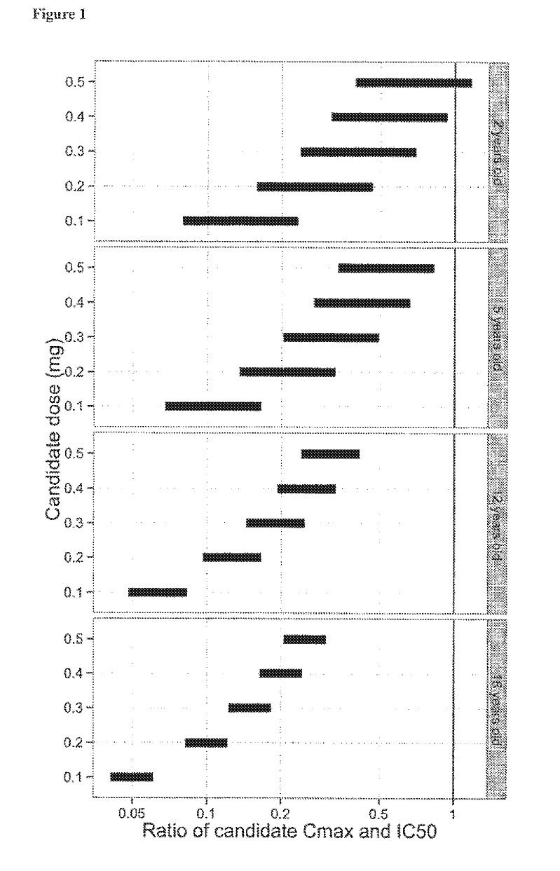

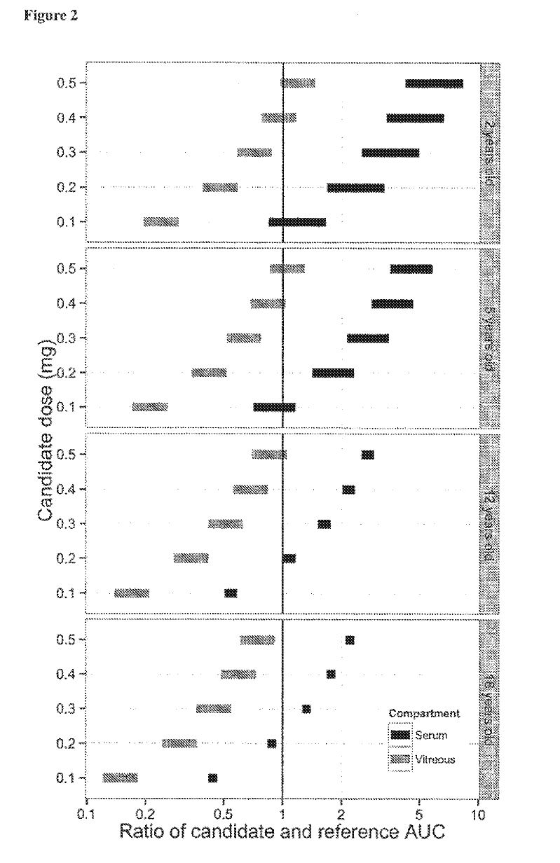

The exposure ratio of Cmax in serum relative to the in vitro IC₅₀ was determined to be less than 1 at all doses of intravitreally administered ranibizumab in all age groups (see FIG. 1). When choosing specific doses for the administration to children, the possibility of underexposure relative to the reference adult vitreal exposure (decreased efficacy) needs to be balanced against the increased serum exposure (increased risk). Age-adjusted doses of 0.2 mg for 2-4 year old children, 0.3 mg for 5-11 year old children and 0.5 mg for 12-17 year old children achieved similar overexposure in serum (ratios of the AUC>1) and similar underexposure in the vitreous (ratios of the AUC<1) using the model described in Example 1 (see FIG. 2). This suggests that these doses may have an appropriate benefit-risk profile on the basis of a clinical interpretation of the predicted exposure ratios.

Dose adjustment for VEGF antagonists other than ranibizumab for the treatment of children can be determined using the predicted ocular and systemic exposure data of ranibizumab described herein.

### Example 3

Forty-five eyes of thirty-nine pediatric patients with choroidal neovascularization (CNV) were treated with intravitreal injection of anti-angiogenic agents (1.25 mg/0.05 ml bevacizumab [40 eyes] or 0.5 mg/0.05 ml ranibizumab [5 eyes]). Choroidal neovascularization due to various causes was clinically diagnosed and confirmed with imaging studies.

There were 24 females and 15 males with group median age 13 years (range 3-17 years). Mean follow-up period was 12.8 months (range 3-60 months). The etiology of the CNV included idiopathic, uveitic, myopic CNV, and CNV associated with various macular dystrophies. Median log MAR visual acuity at presentation and last follow-up was 0.87 (Snellen equivalent 20/150) and 0.7 (Snellen equivalent 20/100), respectively which was statistically significant (p=0.0003). Mean and median number of injections received over the follow-up period was 2.2 and 1, respectively. At the last follow-up, 22 eyes of this group (48%) gained more than 3 lines of vision and 27 eyes (60%) had final visual acuity 20/50 or better. Nine eyes (20%) did not improve and had severe vision loss (20/200 or worse).

Intravitreal anti-angiogenic therapy for CNV in pediatric patients seems temporarily safe and effective in the majority of affected eyes.

### Example 5

A 13-year-old girl was presented with decreased visual acuity of her left eye and optic nerve drusen confirmed by B-scan ultrasound examination in both eyes. Fluorescein angiography and optical coherence tomography revealed the presence of choroidal neovascularization in the left eye. Her best corrected visual acuity was 20/50 in the left eye and 20/25 in the right eye. She demonstrated +8.5 Dsph hyperopia and +0.5 Dcyl astigmatism in both eyes.

The patient was treated with a single injection of ranibizumab (under general anaesthesia) and monitored by clinical examination, optical coherence tomography and fluorescein angiography.

One month after the injection, visual acuity improved from 20/50 to 20/25, central macular thickness was reduced, and sub- and intraretinal fluid was partially resorbed, which was confirmed by OCT. Two months after the injection the visual acuity improved to 20/20. Ophthalmoscopy and OCT showed a complete resolution of the subretinal fluid and macular edema. The fibrotic tissue located between the optic disc and the macula is visible in fluorescein angiography with no signs of activity and recurrence of CNV. 30 months following the injection, the patient's vision remains stable at 20/20, and the macular appearance is stable without the recurrence of subretinal fluid.

Optic nerve drusen should be taken into account and carefully observed as a possible cause of peripapillary choroidal neovascularization in children. Ranibizumab can be a successful off-label treatment in children suffering from choroidal neovascularization associated with optic nerve drusen.

### Example 6

Two cases of idiopathic choroidal neovascularization (CNV) in pediatric patients were treated with intravitreal ranibizumab injections (IVRs).

Case 1

A nine-year old girl was referred for an evaluation of decreased vision in her left eye over several months. The best-corrected visual acuity (BCVA) was 20/20 (OD) and 20/100 (OS). The past medical and ophthalmologic histories were unremarkable and the intraocular pressure (IOP) was 15 mmHg bilaterally. A slit-lamp examination was within normal limits. The fundus examination OD was unremarkable. The left fundus showed a yellow macular elevation with suspected subfoveal haemorrhage. FAG demonstrated a relatively welldefined hyperfluorescent area corresponding to the choroidal neovascularization membrane (CNVM) with late leakage of the dye, including chorioretinal anastomosis, which implied a late stage of classic CNVM. The OCT revealed subfoveal CNVM with high reflectivity, as well as neurosensory detachment, consistent with classic CNVM.

Under topical anesthesia, ranibizumab (0.05 cc-0.5 mg/0.05 mL) was injected supratemporally 3.5 mm posterior to the limbus. One month after IVR, the leakage was decreased, although minimal leakage was suspected during the late phase of FAG. One month after the second injection, the OCT revealed a reduction of subretinal fluid. Two months after the second IVR, the BCVA improved to 20/30 and the CNVM was stained without leakage on FAG. The visual acuity and the lesion were stabilized without any signs of progression or adverse events 14 months after the second IVR. The serologic tests for rubella IgG and herpes simplex IgG were positive; all other serologic tests were negative, but positive serological results were not related to the CNV in the patient.

Case 2

A 10-year-old girl presented with a one-month history of blurred vision in her right eye. The BCVA was 20/50 (OD) and 20/20 (OS). The medical and ophthalmologic histories were unremarkable. A slit-lamp examination and TOP were within normal limits. On dilated fundoscopy, OS was normal; however, OD had a well-defined yellow subretinal exudates with retinal hemorrhage and subretinal fluid, which was consistent with a classic CNV, and subsequently confirmed by OCT and FAG. A ten-year-old girl presented with decreased visual acuity (20/50) in the right eye. The OCT and FAG showed classic CNV. After one IVR, the visual acuity improved to 20/40 and the central foveal thickness was decreased. Visual acuity, FAG, ICG, OCT, serologic tests, and occurrence of ocular or systemic adverse events during follow-up were evaluated.

Under topical anesthesia, ranibizumab (0.05 cc-0.5 mg/0.05 mL) was injected supratemporally 3.5 mm posterior to the limbus. Two months after the first IVR, the FAG revealed that the lesion was stained by the dye without leakage, and the BCVA improved to 20/40 with decreased macular thickness. The BCVA was stabilized, and no serious ocular or systemic adverse events were recorded during 12 months of follow-up. The serologic tests for rubella IgG, toxoplasma IgG, and herpes simplex IgG were positive; the other serologic tests, including toxoplasma IgM, were all negative. However, positive serological results were not related to the CNV in the patient.

### CONCLUSIONS

During 14 and 12 months of follow-up for cases 1 and 2, respectively, no evidence of recurrence or adverse events were noted. The current cases suggest that IVR could be effective in children with idiopathic CNV.

### Example 7

A 13-year-old girl was admitted, complaining of decreased visual acuity in her right eye (RE) for 6 weeks. The best-corrected visual acuity (BCVA) was 20/80 in the RE and 20/20 in the left eye (LE). Ocular and systemic history was unremarkable. Anterior segment examination and intraocular pressure measurements were normal in both eyes. Dilated fundus examination revealed elevated optic discs with blurred margins in both eyes. In addition, an elevated yellow lesion extending from the optic nerve head towards the macula was observed in the RE. Fundus autofluorescence imaging demonstrated bright nodular autofluorescence corresponding to Optic nerve head drusen (ONHD) on the surface of optic nerve head in both eyes. In the RE, a central area of relative hypoautofluorescence surrounded by marked hypoautofluorescence due to CNV and/or subretinal fluid/fibrinous exudate was located at the temporal side of the optic nerve head. The late phase of fluorescein angiography scan showed a central area of hyperfluorescence corresponding to CNV surrounded by blocked fluorescence from subretinal fluid/fibrinous exudate in the RE. Spectral domain optical coherence tomography (SD-OCT) imaging showed irregular bulges over the area of optic nerve head in both eyes. A cross-sectional SD-OCT scan of the macula showed juxtapapillary CNV with high reflectivity and subretinal fluid extending from the optic nerve head to the macula in the RE.

A 0.5 mg/0.05 mL intravitreal ranibizumab injection was then given under general anaesthesia. One month post injection, BCVA increased to 20/25. Serial scans of SD-OCT at months 1, 3 and 9 showed no subretinal fluid. BCVA maintained at the same level (20/25) and no complication related to the injection was observed.

### Example 8

Male and female patients, 12 years and older, are enrolled in a 12-month, randomized, double-masked, sham-controlled, multicenter study evaluating the efficacy and safety of 0.5 mg ranibizumab intravtitreal injections in patients with visual impairment due to VEGF-driven macular edema.

Patients who are diagnosed active ME secondary to any causes (for adult patients: except DME and RVO) are included in the study. Patients are naïve (have not received any prior medication/treatment for the ME lesion under study). BCVA must be between ≥24 and ≤83 letters tested at 4 meters starting distance using ETDRS-like visual acuity charts. Visual loss should be only due to the presence of any eligible types of ME based on ocular clinical, as well as FA and OCT findings.

Women of child-bearing potential, defined as all women physiologically capable of becoming pregnant, unless they are using effective methods of contraception during dosing of study treatment are excluded from the study. In addition, patients are excluded who (i) have history of malignancy of any organ system within the past 5 years; (ii) have history of stroke less than 6 months prior to screening; (iii) have active systemic inflammation or infection, related directly to the underlying causal disease of ME at screening; (iv) have active diabetic retinopathy, active ocular/periocular infectious disease or active intraocular inflammation at screening; (v) have confirmed intraocular pressure (IOP) ≥25 mmHg for any reason at screening; (vi) have neovascularization of the iris or neovascular glaucoma at screening; (vii) have ME secondary to DME or RVO (for adult patients only); (viii) use of any systemic anti-VEGF drugs within 6 months before baseline; (ix) have history of focal/grid laser photocoagulation with involvement of the macular area administered to treat ME at any time; (x) have history of intraocular treatment with any anti-angiogenic drugs (including any anti-VEGF agents) or verteporfin photodynamic therapy (vPDT) at any time; (xi) have history of intravitreal treatment with corticosteroids at any time; (xii) have history of vitreoretinal surgery at any time.

Patients are randomized into two treatment groups:
(1) Patients in the sham control group do not receive active drug. The sham vial does not contain active drug (empty sterile vial). The sham injection is an imitation of an intravitreal injection using an injection syringe without a needle touching the eye. The sham is administered to the patient by the unmasked treating investigator, at the study site, based on a treatment decision made by the masked evaluating investigator. Sham injection is given at baseline, followed by an individualized treatment regimen based on evidence of disease activity assessed at each individual visit as judged and assessed by the investigator. At Month 2, all adult patients randomized into the sham arm will be switched to open-label treatment with ranibizumab, where individualized treatment continues, based on evidence of disease activity.
(2) Patents in the ranibizumab treatment group receive intravitreal injections of ranibizumab, administered by the unmasked treating investigator, at the study site, based on a treatment decision made by the masked evaluating investigator. Ranibizumab 0.5 mg/0.5 mL intravitreal injection is provided as investigational treatment (ranibizumab for intravitreal injection vial in the concentration of 10 mg/mL corresponding to a 0.5 mg dose level). A 0.5 mg ranibizumab intravitreal injection is given to the study eye at baseline followed by further administration of ranibizumab as needed at the follow up study visits, based on evidence of disease activity assessed at each individual visit and as judged by the clinical investigator.

The primary endpoint of the study will be an assessment of Best-corrected visual acuity (BCVA) change from baseline to Month 2 in study eye. Secondary outcome measures are (i) BCVA change from baseline by visit up to Month 2 in study eye (ranibizumab as compared to sham treatment); (ii) change in central subfield thickness (CSFT) and central subfield volume (CSFV) in study eye from baseline over time to Month 2 (assessed by optical coherence tomography (OCT)); (iii) presence of intra-/subretinal fluid in study eye at Month 2 (assessed by OCT images); (iv) presence of active ME leakage assessed by fluorescein angiography (FA) at Month 2 (assessed by photography imaging); (v) requirement for rescue treatment at Month 1; (vi) average BCVA change in study eye from baseline to Month 1 through Month 12 (assessed at baseline, month 1, month 6, month 12; all monthly BCVA outcomes compared to the BCVA at baseline); (vii) change from baseline in CSFT and CSFV in study eye by visit (assessed by OCT at baseline, months 1, 2, 3, 4, 5, 6, 7, 8, 9, 10, 11, 12); (viii) presence of intra-/subretinal fluid in study eye at month 2, month 6, and month 12 compared to baseline (assessed by OCT); (ix) presence of active ME leakage in study eye at month 2, month 6, and month 12 compared to baseline (assessed by OCT); (x) presence of active ME leakage in study eye at month 2, month 6, and month 12 compared to baseline (assessed by photographic images (i.e. Fluorescein angiography)); (xi) proportion of patients with ≥1, ≥5, ≥10 and ≥15 letters gain or reaching 84 letters, at month 2, month 6 and month 12 (this outcome measure represents the proportion of different levels of BCVA gain); (xii) porportion of patients with >1, >5, >10 and >15 letters loss at month 2, month 6 and month 12 (this outcome measure represents the proportion of different levels of BCVA loss); (xiii) number of ranibizumab treatments and re-treatments to study eye by month 2, month 6, month 12 (total number of injections and number of injections given to the study eye by visit); (xiv) type, frequency and severity of ocular and non-ocular adverse events in the study eye up month 2, up to month 6 and up to month 12.

### Example 9

Male and female patients, 12 years and older, are enrolled in a 12-month, randomized, double-masked, sham-controlled, multicenter study evaluating the efficacy and safety of 0.5 mg ranibizumab intravtitreal injections in patients with visual impairment due to VEGF-driven choroidal neovascularization.

Patients who are diagnosed active CNV secondary to any causes, except wAMD and PM in adults are included in the study. All types of CNV lesions present in the study eye. Patients are naïve (have not received any prior medication/treatment for the CNV lesion under study). BCVA must be between ≥24 and ≤83 letters tested at 4 meters starting distance using ETDRS-like visual acuity charts. Visual loss should be only due to the presence of any eligible types of CNV based on ocular clinical, as well as FA.

Women of child-bearing potential, defined as all women physiologically capable of becoming pregnant, unless they are using effective methods of contraception during dosing of study treatment are excluded from the study. In addition, patients are excluded who (i) have history of malignancy of any organ system within the past 5 years; (ii) have history of stroke less than 6 months prior to screening; (iii) active systemic inflammation or infection, related directly to the underlying causal disease of CNV at screening; (iv) have active diabetic retinopathy, active ocular/periocular infectious disease or active intraocular inflammation at screening; (v) have confirmed intraocular pressure ≥25 mmHg for any reason at screening; (vi) have neovascularization of the iris or neovascular glaucoma at screening; (vii) have CNV secondary to PM or wAMD; (viii) use of any systemic anti-VEGF drugs within 6 months before baseline; (ix) have history of focal laser photocoagulation with involvement of the macular area administered to treat CNV at any time; (x) have history of intraocular treatment with any anti-angiogenic drugs or verteporfin photodynamic therapy at any time; (xi) have history of intravitreal treatment with corticosteroids at any time; (xii) have history of vitreoretinal surgery at any time. Furthermore, other protocol-defined inclusion/exclusion criteria may apply.

Patients are randomized to two treatment groups:
(1) Patients in the sham control group do not receive active drug. The sham vial does not contain active drug (empty sterile vial). The sham injection is an imitation of an intravitreal injection using an injection syringe without a needle touching the eye. The sham is administered to the patient by the unmasked treating investigator, at the study site, based on a treatment decision made by the masked evaluating investigator. Sham injection is given at baseline, followed by an individualized treatment regimen based on evidence of disease activity assessed at each individual visit as judged and assessed by the investigator. At Month 2, all adult patients randomized into the sham arm will be switched to open-label treatment with ranibizumab, where individualized treatment continues, based on evidence of disease activity.
(2) Patents in the ranibizumab treatment group receive intravitreal injections of ranibizumab, administered by the unmasked treating investigator, at the study site, based on a treatment decision made by the masked evaluating investigator. Ranibizumab 0.5 mg intravitreal injection is provided as investigational treatment (ranibizumab for intravitreal injection vial in the concentration of 10 mg/mL corresponding to a 0.5 mg dose level). A 0.5 mg ranibizumab intravitreal injection is given to the study eye at baseline followed by further administration of ranibizumab as needed at the follow study visits, based on evidence of disease activity assessed at each individual visit and as judged by the clinical investigator.

The primary endpoint of the study will be an assessment of Best-corrected visual acuity (BCVA) change from baseline to Month 2 in study eye. Secondary outcome measures are (i) BCVA change from baseline by visit up to Month 2 in study eye (ranibizumab as compared to sham treatment); (ii) change in central subfield thickness (CSFT) and central subfield volume (CSFV) in study eye from baseline over time to Month 2 (assessed by optical coherence tomography (OCT)); (iii) presence of intra-/subretinal fluid in study eye at Month 2 (assessed by OCT images); (iv) presence of active chorioretinal leakage assessed by fluorescein angiography (FA) at Month 2 (assessed by photography imaging); (v) average BCVA change in study eye from baseline to Month 1 through Month 12 (assessed at baseline, month 1, month 6, month 12; all monthly BCVA outcomes compared to the BCVA at baseline); (vi) change from baseline in CSFT and CSFV in study eye by visit (assessed by OCT at baseline, months 1, 2, 3, 4, 5, 6, 7, 8, 9, 10, 11, 12); (vii) presence of intra-/subretinal fluid in study eye at month 2, month 6, and month 12 compared to baseline (assessed by OCT); (viii) presence of active chorioretinal leakage in study eye at month 2, month 6, and month 12 compared to baseline (assessed by FA); (ix) proportion of patients with ≥1, ≥5, ≥10 and ≥15 letters gain or reaching 84 letters, at month 2, month 6 and month 12 (this outcome measure represents the proportion of different levels of BCVA gain); (x) porportion of patients with >1, >5, >10 and >15 letters loss at month 2, month 6 and month 12 (this outcome measure represents the proportion of different levels of BCVA loss); (xi) number of ranibizumab treatments and re-treatments to study eye by month 2, month 6, month 12 (total number of injections and number of injections given to the study eye by visit); (xii) type, frequency and severity of ocular and non-ocular adverse events in the study eye up month 2, up to month 6 and up to month 12; (xiii) requirement for rescue treatment at Month 1.

It will be understood that the invention has been described by way of example only and modifications may be made whilst remaining within the scope and spirit of the invention.

1. A method for treating a child having a chorioretinal neovascular or permeability disorder comprising administering to an eye of said child a VEGF antagonist that either does not enter or is rapidly cleared from the child's systemic circulation.

2. The method of claim 1, wherein the VEGF antagonist is ranibizumab.

3. The method of claim 1, wherein the child is below the age of 18 and above the age of 1 year.

4. The method of claim 1, wherein the child is below the age of 12 and above the age of 1 year.

5. The method of claim 3, wherein the dose of the VEGF antagonist administered to the child is the same as the dose typically administered to an adult receiving treatment for a chorioretinal neovascular or permeability disorder.

6. The method of claim 3, wherein the dose of the VEGF antagonist administered to the child is 60% or less of the dose typically administered to an adult receiving treatment for a chorioretinal neovascular or permeability disorder.

7. The method of claim 1, wherein the child is below the age of 5 and above the age of 1 year.

8. The method of claim 7, wherein the dose of the VEGF antagonist administered to the child is 40% or less of the dose typically administered to an adult receiving treatment for a chorioretinal neovascular or permeability disorder.

9. The method of claim 1 comprising administering a first dose of the VEGF antagonist, wherein a second dose of the VEGF antagonist is administered as needed but at least 4 weeks after the first injection.

10. The method of claim 9, wherein the second dose is administered only when continued or recurring disease activity is observed after administration of the first dose.

11. The method of claim 1, wherein the chorioretinal neovascular disorder is secondary to a disease causing inflammation.

12. The method of claim 11, wherein inflammation is caused by the presence of an infectious agent.

13. The method of claim 12, wherein the infectious agent is selected from a virus, a bacterium, a protozoan, a fungus, and a roundworm.

14. The method of claim 1, wherein the method further comprises administering laser photocoagulation therapy (LPT) or photodynamic therapy (PDT).

15. The method of claim 14, wherein initiation of LPT or PDT and of VEGF antagonist administration occur within 1 month of each other.

16. The method of claim 14, wherein initiation of VEGF antagonist administration occurs before initiation of LPT or PDT.

17. The method of claim 1, wherein the method further comprises administering an anti-inflammatory agent.


SEQUENCE LISTING (3 sequences)

SEQ ID NO: 1 - protein, 431 aa, Artificial Sequence
  aflibercept
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 552 aa, Artificial Sequence
  conbercept
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRLPAKYLGYPPPEIKWYKNGIPLESNHTIKAGHVLTIMEVSERDTGNYTVILTNPISKEKQSHVVSLVVYVPPGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 3 - protein, 126 aa, Artificial Sequence
  DARPin MP0112
GSDLGKKLLEAARAGQDDEVRILMANGADVNTADSTGWTPLHLAVPWGHLEIVEVLLKYGADVNAKDFQGWTPLHLAAAIGHQEIVEVLLKNGADVNAQDKFGKTAFDISIDNGNEDLAEILQKAA